(19) United States
(12) Patent Application Publication
(10) Pub. No.: US 2010/0041078 A1
(43) Pub. Date: Feb. 18, 2010

(54) ANTIBODY-BASED GAMMA-HYDROXYBUTYRATE (GHB) DETECTION METHOD AND DEVICE

(75) Inventors: Kestutis G. Bendinskas, Oswego, NY (US); Tia Hendershott, LeRoy, NY (US); James A. MacKenzie, Oswego, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: The Research Foundation of State University of New York, Albany, NY (US)

(21) Appl. No.: 12/522,652

(22) PCT Filed: Jan. 10, 2008
(86) PCT No.: PCT/US08/00375

§ 371 Date: Oct. 27, 2009

Related U.S. Application Data
(60) Provisional application No. 60/879,655, filed on Jan. 10, 2007.

(51) Int. Cl. G01N 33/53; G01N 33/566; C07K 16/00

(52) U.S. Cl. 435/7.92; 436/501; 530/388.1; 530/389.1

(57) ABSTRACT

Gamma-hydroxybutyrate (GHB) can be used as a recreational party drug, aphrodisiac, and attenuator of other drugs and make a person a vulnerable target of robbery or rape. The present invention provides methods and kits for detection of GHB in a sample using an antibody-based assay. Antibodies that specifically bind to GHB or the conjugates of GHB and its derivatives to larger molecules and methods for detecting GHB or its derivatives in bodily fluids and non-alcoholic and alcoholic drinks by employing such antibodies in ELISA or RIA assays are provided by the present invention.

### FIELD OF INVENTION

The present invention relates to antibody-based assays. In particular, the present invention relates to the detection of gamma-hydroxybutyrate (GHB) in a sample using an antibody-based assay. Antibodies that specifically bind to GHB and methods for the production of such antibodies and methods for detecting GHB in bodily fluids and non-alcoholic and alcoholic drinks by employing such antibodies in enzyme-linked immunosorbent assays (ELISA) and radioimmunoassays (RIA) are provided by the present invention.

### BACKGROUND OF THE INVENTION

Gamma-hydroxybutyrate (GHB) is colorless, almost tasteless, and without significant odor, which can be used as a recreational party drug, aphrodisiac, and attenuator of other drugs (Wong et al., 3^{rd} Tr. In Pharm. Sc., 2004, 25 (1), 29-34). GHB is found naturally in the body at extremely low amounts ranging from 5 μg/ml to 10 μg/ml in blood and urine, respectively, and is unique in its ability to cross the blood brain barrier where it can interfere with GABA, gamma-aminobutyric acid, receptors (Elian, Determination of endogenous GHB levels in antemortem urine and blood. Forensic Science International 2002, 128: 120-122). Exogenous administration of GHB can significantly interfere with the receptors in the brain, resulting in neurological and physical side effects. At relatively low amounts, these side effects include temporary amnesia and sleep, which are desirable to sexual predators. At high amounts of exogenous GHB, side effects include coma and death (Crunelli et al. Unraveling the brain targets of GHB. Current Opinion in Pharmacology 2006, 6: 44-52; Struys et al. Metabolism of gamma-hydroxybutyrate to D-2-hydroxglutamate in mammals: further evidence for D-2-hydroxglutamate transhydrogenase. Metabolism Clinical and Experimental 2006, 55: 353-358; Wellendorph et al. Novel Cyclic GHB analogs with high affinity and stereoselectivity of biding to GHB sites in rat brain. The Journal of Pharmacology and Experimental Therapeutics 2005 and Carter et al. Novel GHB analogs share some but not all of the behavioral effects of GHB and GABAb receptor agonists. The Journal of Pharmacology and Experimental Therapeutics 2005, 313(3): 1314-1323).

GHB can make a person a vulnerable target of robbery or rape. Indeed, GHB has been used in substance-related rape cases. Administration of GHB to victims most commonly occurs through beverages, typically in a bar, club, or party scene. After having GHB slipped into their drink, date-rape victims may have a feeling of paralysis and inability to resist, and often, they have no memory of the event (Stillwell, Forensic Sciences, 2002, 47, 5, 1133-1134). A victim of GHB administration may experience decreased inhibitions and increased promiscuity, which can lead to risky behaviors such as use of multiple drugs, failure to use condoms, or participating in relations with strangers. As a result, the chance of contracting sexually transmitted diseases, such as HIV, significantly increases (Wu et al., Concurrent use of methamphetamine, MDMA, LSD, ketamine, GHB, and flunitrazepan among young American youths. Drug and Alcohol Dependence 2006 Sep. 1; 84(1):102-13. Epub 2006 Feb. 17. Erratum in: Drug and Alcohol Dependence 2007 Jan. 12; 86(2-3): 301). There also have been reports of drastic GHB withdrawal symptoms and numerous GHB-related deaths (McDonough et al., Drug and Alcohol Dpnd., 2004, 75, 1, 3-9; Duer et al., Analytical Toxicology, 2001, 25, 7, 576-582). The mechanisms of GHB action are actively studied, which involve GHB and GABA(B) receptors (Maitre et al., Medicine Sciences: M/S, 2005, 21, 3, 284-289).

The current methods for detecting GHB either are expensive, use hazardous reagents, do not use a single-step procedure in drinks containing ethyl alcohol (Bravo et al., Journal of Forensic Sciences, 2004, 49, 2, 379-387), or do not work in various common drinks (e.g. Drink Safe® kit). Current methods of detection include capillary electrophoresis, GC-MS, and LC-MS. Though these methods have experimental data to prove that they work, they are not practical for the use in hospitals and local forensic laboratories. For example, Bortolotti et al. described a method using capillary electrophoresis, which method requires the use of PACE MDQ capillary electrograph and fused silica capillaries (Bortolotti et al. Determination of GHB in biological fluids by using capillary electrophoresis with indirect detection. Journal of Chromatography B 2004, 800: 239-244). Another commonly used method involves the use of gas chromatography-mass spectrometry. This method requires a gas chromatograph with capillary column and mass selective detector for qualitative analysis. In addition, the controls for both groups produced results that were only about −20% accurate. See Elian, GC-MS determination of gamma-hydroxybutyric acid in blood. Forensic Science International 2001, 122: 43-47 and Villain et al. Ultra-rapid procedure to test for the GHB in blood and urine by GC-MS. Journal of Chromatography B 2003, 792: 83-87.

Wood et al. worked with liquid chromatography-tandem mass spectrometry (Wood et al. Simultaneous analysis of GHB and its precursors in urine using liquid chromatography-tandem mass spectrometry. Journal of Chromatography A 2004, 1056: 83-90). Their method requires an LC-MS-MS Alliance system, dC18 column, and preconditioned OASIS MCX solid phase extraction cartridge. Like the GC-MS method, the LC-MS produces results that are only about 20% accurate. Though all of the currently used methods have been shown to detect levels of GHB, they are not practical for use in hospitals and by non-professionals and/or require expensive equipment for detection. In addition, if samples were sent to a central facility, the results would not return in a timely fashion.

Immunodiagnostics is a diagnostic methodology that uses an antigen-antibody reaction as their primary means of detection. The enzyme-linked immunosorbent assay (ELISA, sometimes also called an EIA) is currently one of the predominant formats in immunodiagnostics. The ELISA is a sensitive, rapid, and inexpensive assay, which can be used by professionals and nonprofessionals. The ELISA technique involves the use of antibodies coupled with indicators (e.g., enzymes reacting to produce dyes) to detect the presence of specific substances, such as proteins, viruses, bacteria, or more rarely, smaller molecules.

While there are several different types, basic ELISA utilizes antibodies specific to the antigen being detected by either labeling the antigen or the antibody with an enzyme. There are two different types of ELISA that can be developed. The first is referred to as indirect. Indirect ELISAs utilize a fixed antigen and enzyme labeled antibodies (specific to the primary antibody) to detect for the presence of primary antibodies against the antigen. This form of ELISA has been used for detection of the HIV antibody. The second is direct ELISA, which is based on fixed antibodies and enzyme labeled antibodies, both specific towards the antigen. The direct ELISA is often described as a “sandwich” method that detects the presence of antigens in bodily fluids. The direct ELISA method is typically seen in pregnancy tests. The radioimmunoassays (RIA) are also used in detection of antigens. In this technique, the antigen's concentration is determined indirectly by the competition assay with the radioactively labeled standard for binding to the antibody. However, currently there are no antibodies against GHB that are readily available to be used in an ELISA or RIA assays for the detection of GHB.

U.S. Pat. No. 6,153,147 describes a GHB detection which uses a non-specific indicator and a pen-like housing for the chemical reagent test chamber that is to be dipped in solution with GHB. The disadvantage of such approach is clear non-specificity will yield too many false positives. U.S. Pat. No. 6,156,431 describes a method of GHB extraction followed by its chemical modification and GC-MS analysis. This is one of the traditional approaches, and it involves the use of expensive instrumentation. A method of GHB detection using an enzymatic assay in dry chemistry dipsticks and test pads is described by U.S. Pat. No. 6,617,123. A similar method, using a two-enzyme combination was provided by U.S. Pat. No. 6,703,216. These enzymatic methods are not widely used because enzymes, even though quite specific, are not absolutely specific, usually do not work in ethanol-containing (alcoholic) or acid-containing (e.g. orange juice) solutions as a single step procedure, require specific knowledge of handling of the test, that might not be common in every hospital. In addition, enzymes are notoriously hard to store in their active form. U.S. Pat. No. 6,620,626 describes an approach by which GHB could be detected if GHB-specific antibodies existed. However, U.S. Pat. No. 6,620,626 does not contemplate how to make such antibodies. It simply describes a semipermeable casing that includes such matching antibodies and tiny colored beads. The method described in U.S. Pat. No. 6,620,626 is not likely to be employed in hospitals that rely quite heavily on ELISA-based tests. Recently, U.S. Pat. No. 7,052,854 proposes the use of nanostructures, such as nanoparticles and nanotubes, for detection of multiple compounds, including GHB. This approach has no definite plan or means in detecting specifically GHB, the target of the present invention.

There are numerous patents related to GHB use, such as in use of the treatment of narcolepsy (U.S. Pat. No. 6,780,889), but none of them describes methods of detection of GHB that are relevant to the present invention.

GHB metabolism is quite rapid. It has been discovered that GHB is cleared from the blood, even at high concentrations, with a half-life ranging from twenty minutes to one hour. After eight hours, GHB is virtually undetectable (Bortolotti et al. Determination of GHB in biological fluids by using capillary electrophoresis with indirect detection. Journal of Chromatography B 2004, 800: 239-244; Elian, GC-MS determination of gamma-hydroxybutyric acid in blood. Forensic Science International 2001, 122: 43-47). The rapid metabolism of GHB allows only a certain time frame, in which it can be detected, supporting the need for a test that will produce results rapidly. The market therefore demands a new GHB detection method, which is specific, fast, cost-effective, easy to use in alcohol-containing drinks, and can work in various common drinks and bodily fluids, to be used in various settings, such as hospitals, forensic labs, and research labs. Not only would it be valuable for law enforcement and hospital personnel to have methods that work well in a forensic laboratory, it is imperative that they possess small and simple test kits that work well in the field (National Institute of Drug Abuse, GHB detection kits: “NIDA is urgently seeking SBIR grant application . . . .” See http://www.drugabuse.gov/Funding/GHBKits.html, accessed Jan. 6, 2007). An inconspicuous and publicly available, simple method for detecting GHB in drinks before they are consumed would also be extremely valuable. The object of the present invention is to satisfy the needs in the art by developing such an assay for detection of GHB. The present invention identifies an antibody against GHB that can be used in ELISA tests commonly run in hospitals across the country.

### SUMMARY OF THE INVENTION

The present invention recognizes a specific, inexpensive, and fast antibody-based assay method for the detection of gamma-hydroxybutyrate (GHB), which can work in bodily fluids, such as blood and urine, and various alcoholic and non-alcoholic drinks.

In one aspect, the present invention is directed to an isolated or purified antibody that specifically binds or recognizes gamma-hydroxybutyrate (GHB) in a sample. The antibody identified by the present invention is a polyclonal antibody. Preferably, the antibody identified by the present invention is a monoclonal antibody. The sample can be any liquid sample, particularly, a bodily fluid or a drink, more particularly, an ethyl alcohol-containing drink.

In another aspect, the antibody of the present invention specifically binds or recognizes a conjugate of coupling of GHB or its derivative with at least one carrier molecule. Preferably, the carrier molecule is a protein molecule that is larger than the GHB molecule, such as, but not limited to, bovine serum albumin (BSA) or keyhole limpet hemocyanin (KLH).

In still another aspect, the present invention is directed to a method for detecting gamma-hydroxybutyrate (GHB), comprising providing a sample which is suspected of containing GHB, detecting GHB in the sample by performing any of many types of enzyme-linked immunosorbent assays (ELISA) or radioimmunoassays (RIA) using an antibody that specifically binds or recognizes GHB, and determining whether GHB is present in the sample by viewing, measuring or quantifying the resulting signal obtained from ELISA or RIA. The antibody identified by the present invention is a polyclonal antibody. Preferably, the antibody is a monoclonal antibody. In a particular aspect, the method of the present invention is used for detection of GHB in a bodily fluid or a drink sample that comprises alcohol.

In yet another aspect, the present invention provides a kit for detecting GHB in a sample, preferably, a sample containing alcohol. According the present invention, the kit includes at least one of the antibodies as described above and components for performing an ELISA or RIA assay.

In a further aspect, the present invention is directed to a method for producing an antibody that specifically binds or specifically recognizes GHB, comprising conjugation or coupling of at least one carrier molecule to GHB or its derivative to obtain a conjugated or coupled GHB molecule, administering an immunogen containing the conjugated or coupled GHB molecules to an animal, and isolating antibodies that specifically bind or recognize GHB from the animal. According to the present invention, the carrier molecule can be a protein molecule that is larger than GHB molecule, such as, but not limited to, BSA or KLH.

In still another aspect, the present invention provides a device for detecting GHB in a sample. The device is precoated with GHB or its derivative or an antibody that reacts with GHB or its derivative and produces a color change or any other measurable change on the device indicating the presence of GHB in the suspected sample.

### BRIEF DESCRIPTION OF THE DRAWINGS

FIG. 1A shows a typical ELISA, in which the primary antibody is attached to the plate, then GHB in a suspect solution binds non-covalently to the primary antibody, then the secondary antibody binds, and the enzyme attached to it produces the color reaction.

FIG. 1B shows an ELISA in which GHB or its derivative is bound to the plate, then the solution suspected containing GHB is applied, the primary antibody is applied, and then secondary antibody binds, its enzyme providing the color when substrates are added.

FIG. 1C shows an ELISA in which primary antibody is bound to the plate, and the suspect solution with GHB is competing for binding to primary antibody with the GHB conjugated to the reporter enzyme that produces color when substrates are added.

FIG. 1D depicts an RIA, in which primary antibody is bound to the plate, and GHB in the suspect solution is competing for binding to primary antibody with the GHB that is radioactively labeled.

FIG. 2A depicts Indirect ELISA; FIG. 2B depicts Indirect Competitive ELISA.

FIG. 3A depicts Direct ELISA; FIG. 3B depicts Direct Competitive ELISA.

FIG. 4 depicts a GOBAB-KLH conjugated protein produced by crosslinking GOBAB to KLH.

FIG. 5 depicts results of the absorbance for a preliminary test. Blk-Blank, G-GOBAB linked to the plate, BSA-BSA linked to the plate, 1-IgG-Primary antibody from Spring Valley linked to the plate, 2-IgG-Secondary antibody (HRPO from Sigma) linked to the plate, and S/S—Substrate and stop solutions.

FIG. 6 depicts results of the absorbance for competitive GHB and GOBAB. Blk-Blank, G-GOBAB linked to the plate, BSA-BSA linked to the plate, 1 IgG-Primary antibody from Spring Valley linked to the plate, 2 IgG-Secondary antibody (HRPO from Sigma) linked to the plate, S/S—Substrate and stop solutions, A-GOBAB linked to the plate; competition with GOBAB, B-GOBAB linked to the plate; and competition with GHB: 1-50 mM (amount of competitor) and 2-25 mM (amount of competitor)

FIG. 7 depicts GHB competition standard curve.

### DETAILED DESCRIPTION OF THE INVENTION

The present invention is directed to a specific, inexpensive, and fast antibody-based assay method for the detection of gamma-hydroxybutyrate (GHB), which is effective in bodily fluids and various alcoholic and non-alcoholic drinks.

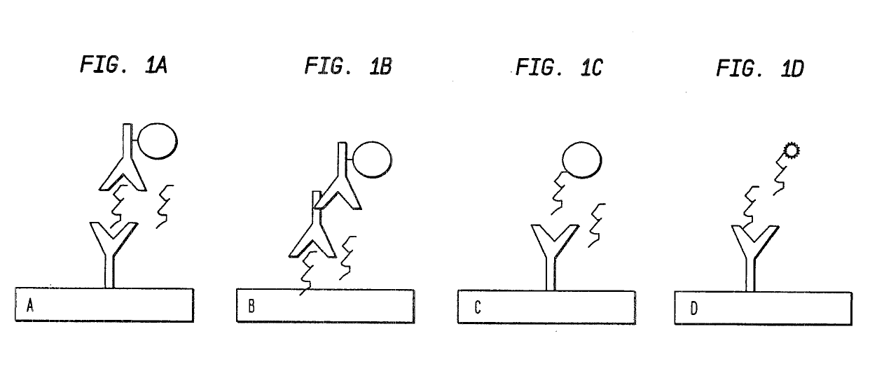

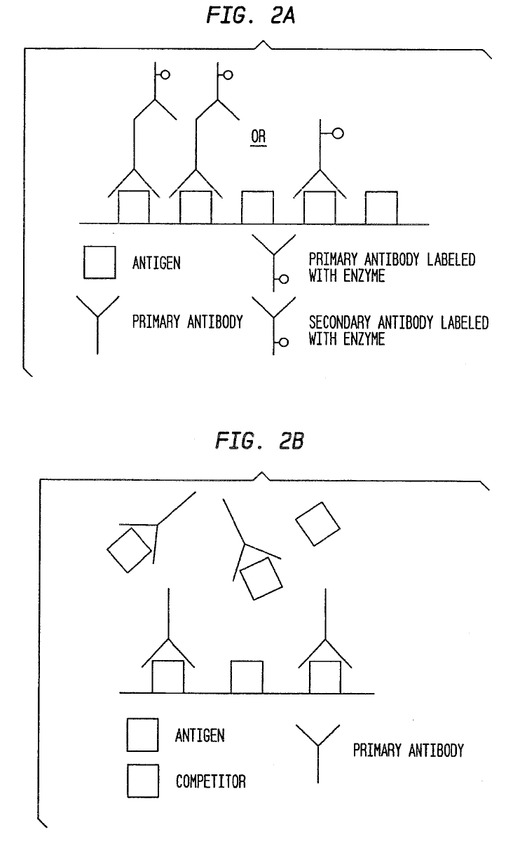

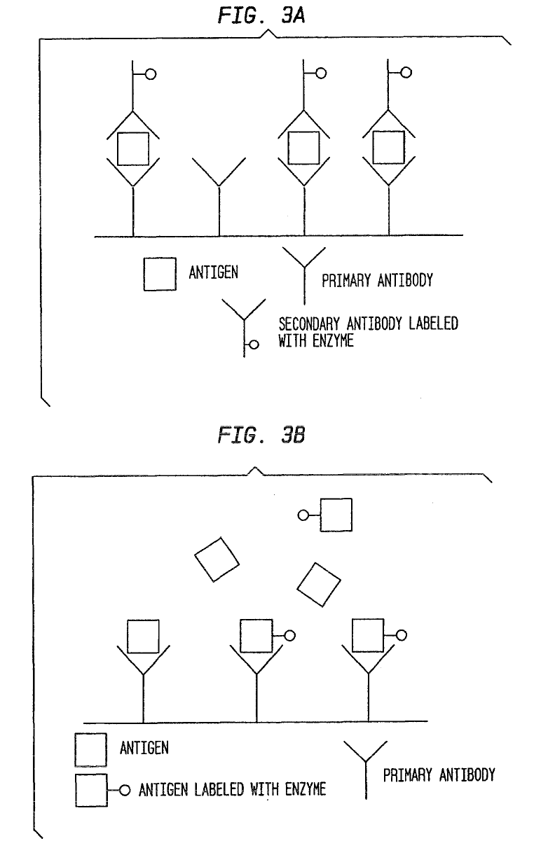

According to the present invention, competitive indirect and competitive direct methods for performing ELISA can be used for detecting GHB. In competitive tests, a known amount of the antigen competes for the antibodies with the unknown amount in a sample (see FIGS. 1A-1D, 2A-2B, and 3A-3B). An indirect method test is exemplified in Example 2. Preferably, the test is further optimized and cross specificity is checked. The indirect test is not as rapid as the direct method. Once the antibodies specific for GHB are confirmed, e.g., by the indirect method of the present invention, a direct method can be tested by crosslinking a primary antibody to the well plate and competing various GHB concentrations with a second antibody, e.g., GHB-horseradish peroxidase conjugate. The direct method test normally takes approximately 1.5 hours or less to complete and is more practical for use in hospital laboratories both economically and medically.

The mass production and use of these innovative tests for GHB in accordance of the present invention have substantial societal and medical benefits. With the availability of a rapid and efficient GHB detection method, ELISA, the diagnosis and further prevention can decrease the illicit use of GHB, which illicit use can cause many side effects or lead to devastating consequences of date rape. In addition, the present invention facilitates detection and/or diagnosis of illicit use of GHB and the side effects thereof, which are useful in the additional research to determine a remedy or antidote to rapidly reverse the side effects of GHB.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

The terms “gamma-hydroxybutyric acid,” “gamma-hydroxybutyrate,” “GHB”, “4-hydroxybutyric acid,” “oxybutyrate” and the like are used interchangeably and refer to the chemical 4-hydroxy-butanoic acid. Also encompassed by these terms are GHB derivatives, analogs, salts, and isomers thereof, which are structurally related to GHB and produce a pharmacological effect like GHB. Examples of such substances include, but are not limited to GOBAB (3-amino-4-hydroxy butyric acid) (a GHB analog), gamma hydroxyburyralactone (GBL), gamma hydroxyvaleric acid (GHV), 1,4-butanediol (BD), and amine, thiol, carboxylic acid, alcohol, aldehyde derivatives of GHB. A variety of colloquial “street names” are also used for GHB, including Soap, Scoop, Max, Liquid Ecstasy, Grievous Bodily Harm, Goop, Georgia Home Boy, Easy Lay, Cherry Meth, Everclear, Fantasy, G, G-riffic, Gamma Oh, GBH, Jib, Liquid E, Organic quaalude, Salty water, Sleep-500, Somatomax, Vita-G, and Water.

By “conjugated” is meant indirect connection, attachment, linkage, or conjugation unless the context clearly dictates otherwise.

By “specifically bind” or “specifically recognize” refers to the increased propensity of one member of a binding pair to bind to a second member of a binding pair as compared to other molecules present in the sample. By “binding pair” refers to first and second molecules that bind specifically to each other with greater affinity than to other components in the sample. The binding between the members of the binding pair is typically noncovalent. Exemplary binding pairs include immunological binding pairs (e.g. any haptenic or antigenic compound in combination with a corresponding antibody or binding portion or fragment thereof, for example digoxigenin and anti-digoxigenin, mouse immunoglobulin and goat anti-mouse immunoglobulin) and nonimmunological binding pairs (e.g., biotin-avidin, biotin-streptavidin, hormone [e.g., thyroxine and cortisol]-hormone binding protein, receptor-receptor agonist or antagonist (e.g., acetylcholine receptor-acetylcholine or an analog thereof), IgG-protein A, lectin-carbohydrate, enzyme—enzyme cofactor, enzyme—enzyme inhibitor, and complementary polynucleotide pairs capable of forming nucleic acid duplexes). One or both members of the binding pair can be conjugated to additional molecules.

“Polypeptide” and “protein” are used interchangeably herein and include a molecular chain of amino acids linked through peptide bonds. The terms do not refer to a specific length of the product. The terms include polypeptides containing co- and/or post-translational modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations or esterifications of carboxyl groups) and the like are included within the meaning of polypeptide.

The term “substrate” refers to a molecule that is a reactant for an enzymatic reaction.

The term “antibody” as used herein includes antibodies obtained from both polyclonal and monoclonal preparations, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, for example, Riechmann et al. (1988) Nature 332:323-327; Verhoeyan et al. (1988) Science 239:1534-1536; and U.K. Patent Publication No. GB 2,276,169, published Sep. 21, 1994); and any functional fragments obtained from such molecules, wherein such fragments retain specific-binding properties of the parent antibody molecule.

By “monoclonal antibody” is meant an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human hybridomas or from murine hybridomas made from mice expressing human immunoglobulin chain genes or portions thereof. See, e.g., Cote, et al. Monoclonal Antibodies and Cancer Therapy, Alan R. Liss, 1985, p. 77.

In one embodiment, the present invention is directed to an isolated or purified antibody that specifically binds or recognizes gamma-hydroxybutyrate (GHB) or derivatives, analogs, salts, and isomers thereof in a sample. The antibody identified by the present invention is a polyclonal antibody. Preferably, the antibody identified by the present invention is a monoclonal antibody. The procedures for isolation and purification of antibodies are well known in the art.

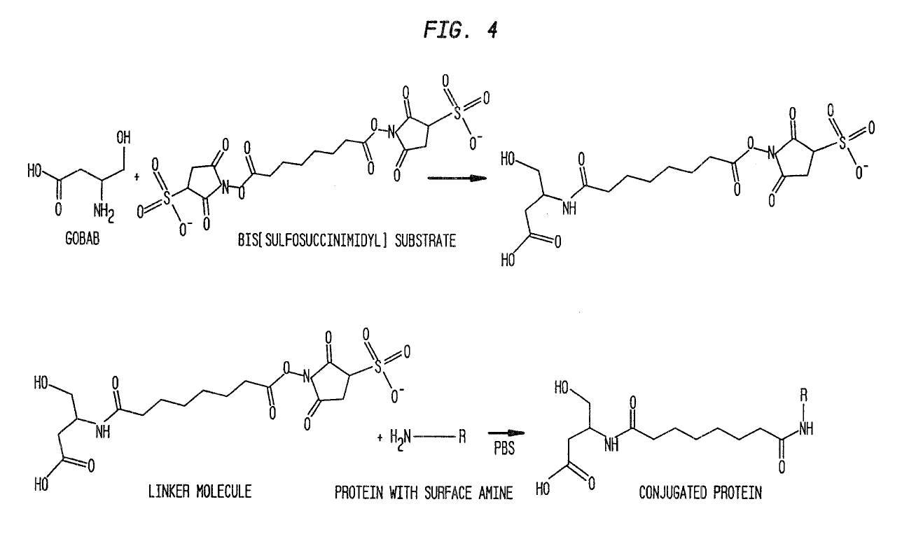

However, successful isolation and purification of desired antibodies depend on each specific antibody on a case-by-case basis. According to the present invention, an antibody to GHB can be generated by injecting GHB or GHB derivative conjugate molecule to an antibody-producing animal, e.g., a mouse or rabbit. Preferably, GHB or GHB derivative is linked or conjugated to one or more carrier molecule, e.g., a protein molecule, for generating antibodies. Examples of carrier molecules include, but are not limited to, bovine serum albumin (BSA) and keyhole limpet hemocyanin (KLH). A GOBAB-KLH conjugated protein, which is produced by crosslinking GOBAB to KLH, is demonstrated in FIG. 4. The resulting conjugated protein can be then used to produce polyclonal antibodies, e.g., by boosting in rabbits.

Accordingly, in another embodiment, the antibody of the present invention specifically binds or recognizes a conjugation or coupling of GHB or its derivative and at least one carrier molecule. Preferably, the carrier molecule is a protein molecule that is larger than GHB molecule, such as, but not limited to, bovine serum albumin (BSA) and keyhole limpet hemocyanin (KLH). The serum from the animal is then tested for antibody activity.

The sample to be assayed can be any liquid sample, particularly, a bodily fluid or a drink, more particularly, an ethyl alcohol-containing sample. Given the degree of abuse of GHB and the short half-life of the compound, trace amounts of GHB in bodily fluids consumed by GHB-related crime-victims are particularly contemplated samples of the present invention.

In still another embodiment, the present invention is directed to a method for detecting GHB, comprising providing a sample, which is suspected of containing GHB, detecting GHB in the sample by performing an ELISA or RIA assay using an antibody that specifically binds or recognizes GHB, and determining whether GHB is present in the sample by viewing or measuring or quantifying the resulting signal obtained from ELISA or RIA. The antibody is a polyclonal antibody. Preferably, however, the antibody is a monoclonal antibody against GHB or a GHB-conjugate. In a particular aspect, the method of the present invention is used for detection of GHB in a sample that comprises alcohol, e.g., an alcoholic beverage consumed by a suspected date-rape victim. ELISA and RIA assay procedures are well known in the art and are further illustrated in the figures described herein.

FIG. 1A shows a typical ELISA, in which the primary antibody is attached to the plate, then GHB in a suspect solution binds non-covalently to the primary antibody, then the secondary antibody binds, and the enzyme attached to it produces the color reaction.

FIG. 1B shows an ELISA in which GHB or its derivative is bound to the plate, then the solution suspected containing GHB is applied, the primary antibody is applied, and then secondary antibody binds, its enzyme providing the color when substrates are added.

FIG. 1C shows an ELISA in which primary antibody is bound to the plate, and the suspect solution with GHB is competing for binding to primary antibody with the GHB conjugated to the reporter enzyme that produces color when substrates are added.

FIG. 1D depicts an RIA, in which primary antibody is bound to the plate, and GHB in the suspect solution is competing for binding to primary antibody with the GHB that is radioactively labeled.

In yet another embodiment, the present invention provides a kit for detecting GHB in a sample, preferably, a sample containing alcohol. According the present invention, the kit includes at least one of the antibodies as described above and components for performing an ELISA or RIA assay. The components for an ELISA or RIA assay can include, but are not limited to, plates for experimental and control ELISA reactions, buffer PBS at appropriate pH, e.g., pH 7.5, TWEEN 20, carbonate buffer at appropriate pH, e.g., pH=9.6, non fetal calf serum, which is stored in frozen state, bovine serum albumin (BSA), goat anti rabbit IgG labeled with horseradish peroxidase, e.g., in tubes of 1 ml and preserved at 4° C., washing buffer (PBS buffer) at appropriate pH, e.g., pH 7.5, dilution buffer, GHB, radioactively labeled GHB, and substrates.

The present invention also contemplates a test strip, test stick, dip stick, stirrer, well plate, or coaster device which can be conventionally coated with GHB or its derivative or an antibody that reacts with GHB or its derivatives and produces a color change or any other detectable or measurable change on the device, indicating the presence of GHB in the suspected sample. The sample may be a bodily fluid, an alcoholic or non-alcoholic drink. The change in color can occur substantially instantaneously, for example, within several seconds or several minutes. The test strip may be of paper construction, like litmus paper. The test stick, dip stick, stirrer, well plate, or coaster device can comprise paper, cardboard, plastic (PVC, PET), wood or the like. Thus, a detection of GHB using antibodies is achieved for the first time in accordance with the present invention.

In a further embodiment, the present invention is directed to a method for producing an antibody that specifically binds or specifically recognizes GHB, comprising the conjugation or coupling of at least one carrier molecule to GHB or GHB derivative to obtain a conjugated or coupled GHB molecule, administering an immunogen containing the conjugated or coupled GHB molecules to an animal, and isolating antibodies that specifically binds or recognizes GHB from the animal. According to the present invention, the carrier molecule can be a molecule that is larger than GHB molecule, such as, but is not limited to, BSA and KLH.

The following examples supplement experimental work described above, and are presented by way of illustration and not by any way of limitation.

### Example 1

3-amino-4-hydroxybutyric (GOBAB) acid was coupled to BSA and KLH using gluteraldehyde (GA) and bis[sulfosuccinimidyl]suberate (BS3) coupling reagents yielding 4 variants of GHB-labeled conjugates. These were dialyzed against PBS to remove the remaining reagents and then tested for conjugation. The GOBAB-BS3-KLH conjugate was chosen for further work. Two rabbits were immunized and the boosts were performed 3, 6, and 9 weeks after the initial immunization. The serum was collected 10 days after each boost. GOBAB was crosslinked to a 96-well polystyrene plate via a maleic anhydride linker anchored in the plate for testing of ELISA via the method as depicted in FIG. 1B. It yielded a positive signal upon addition of primary and secondary antibodies, when the conjugated horseradish peroxidase reacted with its substrates. The signal was significantly larger in the wells with GHB-linked to the well as compared to the control wells that had nothing or BSA linked to the bottom of the well. GOGAB was also crosslinked using BS3 to horseradish peroxidase for testing in ELISA as depicted in FIG. 1C.

### Example 2

In order to produce an ELISA, indirect or direct, antibodies needed to be produced that can select for GHB. However, due to its small size, GHB does not illicit a sufficient immunological response. Therefore, a GHB analog, GOBAB (3-amino-4-hydroxy butyric acid), was chosen to crosslink to a large protein, KLH (keyhole limpet hemocyanin) (see FIG. 4). The resulting conjugated protein was then boosted in two rabbits at Spring Valley Laboratories to produce polyclonal antibodies.

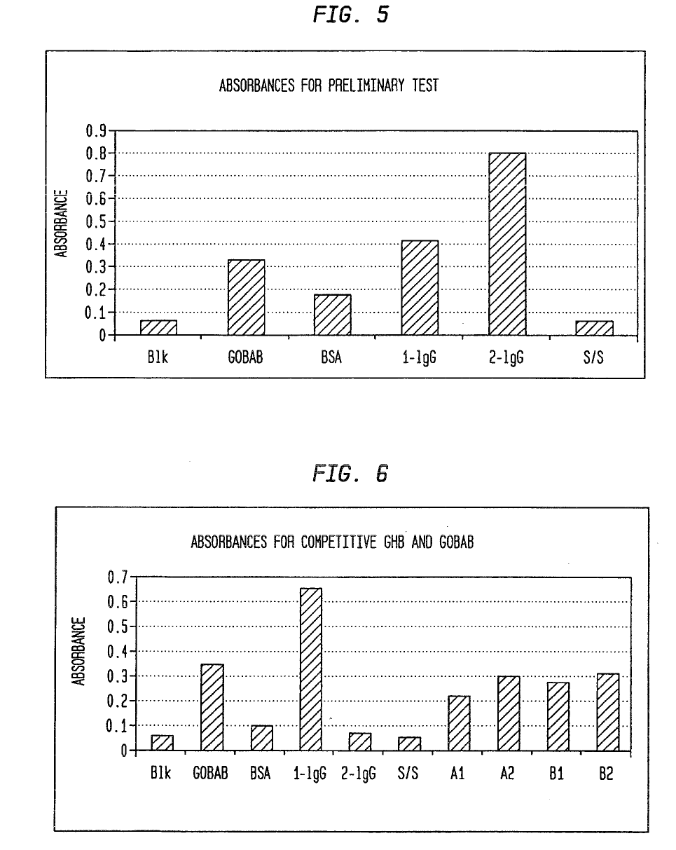

When the antibodies returned from Spring Valley, an indirect ELISA with competition was first developed to determine the ability to detect GOBAB and GHB. Data can be seen in Tables 1-2 and FIGS. 5-6. Experiments 1 and 2 were run in duplicate. The results confirmed that the antibodies detected both GOBAB and GHB.

### Example 3

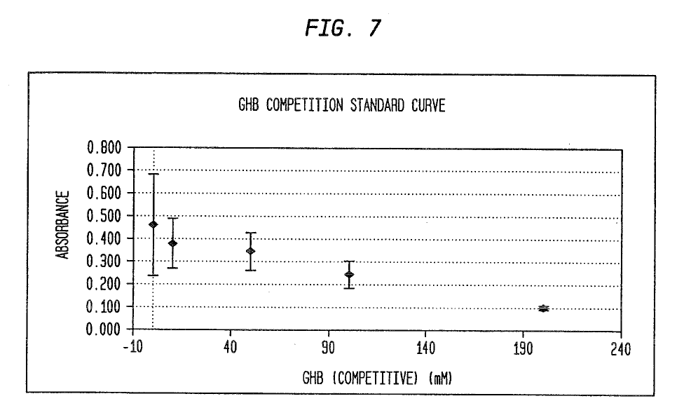

Additional indirect competition tests were performed to develop a standard curve for different GHB concentrations. Competitive amounts of GHB ranged from 10 mM to 200 mM so as to include the cut off point for blood and urine at the low end of the curve and test for the high concentration of GHB used in a date rape scenario. See Table 3 and FIG. 7 (results of Experiment 3), which clearly illustrate that as the competition of GHB increases, the absorbance decreases confirming that the antibodies contemplated by the present invention detect GHB.

TABLE 1
Results of Experiment 1 (the absorbance for a preliminary test).
Template

| | | | | | |
|---|---|---|---|---|---|
| Blk | GOBAB | BSA | 1-IgG | 2-IgG | S/S |
| Blk | GOBAB | BSA | 1-IgG | 2-IgG | S/S |

Data

| | | | | | |
|---|---|---|---|---|---|
| 0.055 | 0.32 | 0.18 | 0.43 | 0.842 | 0.067 |
| 0.058 | 0.333 | 0.17 | 0.401 | 0.756 | 0.05 |

Blk—Blank,
G—GOBAB linked to the plate,
BSA—BSA linked to the plate,
1-IgG - Primary antibody from Spring Valley linked to the plate,
2-IgG - Secondary antibody (HRPO from Sigma) linked to the plate, and
S/S—Substrate and stop solutions.

TABLE 2
Results of Experiment 2 (the absorbance for competitive GHB and
GOBAB)
Template

| | | | | | |
|---|---|---|---|---|---|
| Blk | GOBAB | BSA | 1 IgG | 2 IgG | S/S |
| Blk | GOBAB | BSA | 1 IgG | 2 IgG | S/S |
| A1 | A1 | A1 | B1 | B1 | B1 |
| A2 | A2 | A2 | B2 | B2 | B2 |

Raw Data

| | | | | | |
|---|---|---|---|---|---|
| 0.054 | 0.321 | 0.088 | 0.633 | 0.06 | 0.049 |
| 0.054 | 0.369 | 0.111 | 0.666 | 0.069 | 0.047 |
| 0.182 | 0.221 | 0.248 | 0.311 | 0.249 | 0.249 |
| 0.28 | 0.302 | 0.327 | 0.3 | 0.292 | 0.339 |

Blk—Blank
G—GOBAB linked to the plate
BSA—BSA linked to the plate
1 IgG - Primary antibody from Spring Valley linked to the plate
2 IgG - Secondary antibody (HRPO from Sigma) linked to the plate
S/S—Substrate and stop solutions
A - GOBAB linked to the plate; competition with GOBAB
B - GOBAB linked to the plate; competition with GHB
1-50 mM (amount of competitor)
2-25 mM (amount of competitor)

TABLE 3
Results of Experiment 3 (Indirect ELISA- GHB competition standard curve)
Template

| | | | | | | | | | | | |
|---|---|---|---|---|---|---|---|---|---|---|---|
| BLK | G1 | G2 | B | 1 IgG | 2 IgG | E5 | E6 | E7 | F5 | F6 | F7 |
| BLK | G1 | G2 | B | 1 IgG | 2 IgG | E5 | E6 | E7 | F5 | F6 | F7 |
| BLK | G1 | G2 | B | 1 IgG | 2 IgG | E5 | E6 | E7 | F5 | F6 | F7 |
| A1 | A2 | A3 | A4 | B1 | B2 | B3 | B4 | S/S | | | |
| A1 | A2 | A3 | A4 | B1 | B2 | B3 | B4 | S/S | | | |
| A1 | A2 | A3 | A4 | B1 | B2 | B3 | B4 | S/S | | | |

Raw Data

| | | | | | | | | | | | |
|---|---|---|---|---|---|---|---|---|---|---|---|
| 0.055 | 0.303 | 0.632 | 0.38 | 0.801 | 0.343 | 0.226 | 0.325 | 0.403 | 0.12 | 0.117 | 0.19 |
| 0.054 | 0.281 | 0.539 | 0.336 | 0.858 | 0.567 | 0.305 | 0.291 | 0.322 | 0.13 | 0.101 | 0.155 |
| 0.061 | 0.271 | 0.211 | 0.276 | 0.597 | 0.527 | 0.216 | 0.253 | 0.274 | 0.11 | 0.095 | 0.101 |
| 0.411 | 0.331 | 0.232 | 0.107 | 0.499 | 0.437 | 0.306 | 0.109 | 0.054 | | | |
| 0.322 | 0.305 | 0.219 | 0.101 | 0.312 | 0.311 | 0.226 | 0.102 | 0.058 | | | |
| 0.325 | 0.268 | 0.184 | 0.097 | 0.307 | 0.277 | 0.19 | 0.091 | 0.062 | | | |

| Sample | Ave Abs | SD | GHB Conc (mM) |
|---|---|---|---|
| B0 | 0.461 | 0.221 | 0 |
| B1 | 0.373 | 0.109 | 10 |
| B2 | 0.342 | 0.084 | 50 |
| B3 | 0.241 | 0.059 | 100 |
| B4 | 0.101 | 0.009 | 200 |

Blk—Blank
S/S—Substrate and Stop solution
G1—GOBAB linked to plate (30 min substrate incubation)
G2—GOBAB linked to plate (1 hr substrate incubation)
B—BSA linked to plate
1 IgG—Primary antibody linked to plate
2 IgG—Secondary antibody linked to plate
A—GHB competition test 30 min substrate incubation (GOBAB linked to plate)
B—GHB competition test 1 hr substrate incubation (GOBAB linked to plate)
1—10 mM GHB
2—50 mM GHB
3—100 mM GHB
4—200 mM GHB
E—Ethanol control (GOBAB linked to plate)
F—Ethanol + competition with 200 mM GHB (GOBAB linked to plate)
5—4% Ethanol
6—12% Ethanol
7—40% Ethanol

What is claimed:

1. An isolated or purified antibody that specifically binds or recognizes gamma-hydroxybutyrate (GHB) in a sample.

2. The antibody of claim 1, wherein said antibody is a polyclonal antibody.

3. The antibody of claim 1, wherein said antibody is a monoclonal antibody.

4. The antibody of any one of claims 1-3, wherein the antibody specifically binds or recognizes a conjugation or coupling of GHB or GHB derivative and at least one carrier molecule.

5. The antibody of claim 4, wherein the carrier molecule is a molecule that is larger than GHB molecule.

6. The antibody of claim 5, wherein the protein is bovine serum albumin (BSA) or keyhole limpet hemocyanin (KLH).

7. The antibody of claim 1, wherein the sample comprises alcohol.

8. A method for detecting gamma-hydroxybutyrate (GHB), comprising
a. providing a sample which is suspected of containing GHB,
b. detecting GHB in the sample by performing an ELISA or RIA assay using an antibody that specifically binds or recognizes GHB and a conjugate of GHB or a GHB derivative to a protein molecule, and
c. determining whether GHB is present in the sample by viewing or measuring or quantifying the resulting signal obtained from ELISA or RIA.

9. The method of claim 8, wherein the antibody is a polyclonal antibody.

10. The method of claim 8, wherein the antibody is the monoclonal antibody.

11. The method of any one of claims 8-10, wherein the sample comprises alcohol.

12. The method of claim 8, wherein said protein is bovine serum albumin (BSA) or keyhole limpet hemocyanin (KLH).

13. A kit for detecting GHB in a sample comprising the antibody of claim 1 and components for performing an ELISA or RIA.

14. A method for producing an antibody that specifically binds or recognizes GHB, comprising
a. conjugating or coupling at least one carrier molecule to GHB or GHB derivative,
b. administering an immunogen comprising the conjugated or coupled molecules from Step a to an animal, and
c. isolating an antibody from said animal of Step b that specifically binds or recognizes GHB.

15. The method of claim 14, wherein the carrier molecule is a protein molecule that is larger than said GHB molecule.

16. The method claim 15, wherein the protein is BSA or KLH.

17. A device comprising an isolated or purified antibody that specifically binds or recognizes gamma hydroxybutyrate (GHB) or GHB derivative.

18. The device of claim 17, wherein said device is a constructed of a material selected from the group consisting of paper, plastic, or wood.
